(19) United States
(12) Patent Application Publication
(10) Pub. No.: US 2024/0402112 A1
(43) Pub. Date: Dec. 5, 2024

(54) Method for Operating a Plurality of Electrolyser-Stacks

(71) Applicant: ABB Schweiz AG, Baden (CH)

(72) Inventors: Matthias Biskoping, Hirschberg (DE); Bernhard Primas, Mannheim (DE); Andres Leal-Ayala, Ennetbaden (CH); Georg Gutermuth, Heidelberg (DE); Kai Koenig, Walldorf (DE); Stefan Thorburn, Västerås (SE); Daniel Chartouni, Wettingen (CH)

(73) Assignee: ABB Schweiz AG, Baden (CH)

(21) Appl. No.: 18/799,249

(22) Filed: Aug. 9, 2024

Related U.S. Application Data
(63) Continuation of application No. PCT/EP2023/052990, filed on Feb. 7, 2023.

(30) Foreign Application Priority Data
Feb. 11, 2022 (EP) 22156365.3

(51) Int. Cl. G01N 27/02; C25B 1/04; C25B 9/77; C25B 15/023; G01N 33/00

(52) U.S. Cl. CPC G01N 27/026; C25B 1/04; C25B 9/77; C25B 15/023; G01N 33/0027

(57) ABSTRACT

A method for operating a plurality of electrolyzer-stacks includes determining a concentration of impurities, which is originated by a second reaction gas electrochemically produced at a second electrode type of each of the electrolyzer-stacks, within a first gas stream; generating a trigger signal if the concentration of the impurities of the second reaction gas within the merged first reaction gas exceeds a specific second reaction gas level; identifying at least one electrolyzer-stack out of the plurality of electrolyzer-stacks, which is low performing in respect to excessively feeding second reaction gas impurities into the first gas stream, by measuring a current density of at least one electrolyzer-stack of the plurality of electrolyzer-stacks, if the trigger signal is generated.

### CROSS-REFERENCE TO RELATED APPLICATIONS

The instant application claims priority to International Patent Application No. PCT/EP2023/052990, filed Feb. 7, 2023, and to European Patent Application No. 22156365.3, filed Feb. 11, 2022, each of which is incorporated herein in its entirety by reference.

### FIELD OF THE DISCLOSURE

The instant disclosure generally relates to systems and method for operating electrolyzer stacks and, more particularly, to systems and methods for operating a plurality of electrolyzer stacks.

### BACKGROUND OF THE INVENTION

A number of electrolysis processes have industrial applications. For example, the electrolysis of water provides a source of hydrogen and oxygen. Alternatively, the electrolysis of water containing sodium chloride can provide a source of hydrogen, chlorine, and sodium hydroxide. Water electrolysis processes fundamentally involve the splitting of water into its constituents although other chemistries also utilizing the electrolysis of water are certainly possible.

In a water-electrolyzer water, is split into its components: hydrogen gas and oxygen gas. An electrolyzer essentially comprises a cathode-electrode, an anode-electrode, and an electrolyte. Hydrogen is produced at the cathode via the Hydrogen Evolution Reaction (HER). Oxygen is produced at the anode via the Oxygen Evolution Reaction (OER).

### BRIEF SUMMARY OF THE INVENTION

For operation of a plurality of electrolyzer-stacks it is useful to control a correct performance of each of the electrolyzer-stacks and/or each cell of the electrolyzer-stacks. The invention relates to the field of electrolysis technology and relates to an improved method for operating an electrolysis cell/stack mainly in respect of monitoring failure situations.

Considering a large scale, multi-megawatt hydrogen production facility consisting of dozens of water electrolyzers, it is necessary to have an efficient method to guarantee hydrogen production with enhanced purity and reduced safety risks.

When such an electrolyzer park, including a plurality of electrolyzer-stacks, is operating at full capacity, oxygen and hydrogen separation in the electrolysis cell can be optimal. However, when a decrease in the availability of renewable energy forces the control system to reduce the operating capacity of one or more electrolyzers, pressure gradients in the electrolysis cells can lead to hydrogen diffusion through the membrane to the oxygen side of the cell, or oxygen diffusion to the hydrogen side of the cell.

Hydrogen diffusion to the oxygen half-cell can create an explosive atmosphere if a safety limit of about 2% hydrogen in oxygen is exceeded, while oxygen diffusion into the hydrogen half-cell can reduce the purity of the hydrogen, increasing energy consumption for purification purposes in subsequent process steps, reducing an overall efficiency of the system.

A use of physical hydrogen and oxygen sensors to monitor the situation at every electrolyzer-stack of the plurality of electrolyzer-stacks in a multi MW-facility can end up with the installation of hundreds of sensors. A process requiring hundreds of sensors can be unreliable and can have very high maintenance costs associated with ensuring the accuracy of every sensor.

Accordingly, the present disclosure describes a method for operating a plurality of electrolyzer-stacks, an electrolyzer-stack operation device and a use of an electrolyzer-stack operation device. All combinations of at least two of the features disclosed in the description, the claims and the figures fall within the scope of the invention. In order to avoid repetition, features disclosed in accordance with the method shall also apply and be claimable in accordance with mentioned systems.

In this entire description, the sequence of procedural steps is presented in such a way that the process is easily comprehensible. However, the skilled person will recognize that many of the process steps can also be executed in a different order and lead to the same or a corresponding result. In this sense, the sequence of the process steps can be changed accordingly. Some features are provided with counting words to improve readability or to make the assignment more clear, but this does not imply the presence of certain features.

To achieve these and other advantages and in accordance with the purpose of the invention, as embodied and broadly described herein, there is provided a method for operating a plurality of electrolyzer-stacks, wherein each of the plurality of electrolyzer-stacks are configured to be provided with water and electrical energy to produce at least a first reaction gas electrochemically at a first electrode type of each of the electrolyzer-stacks, and wherein the first reaction gas produced by each of the plurality of electrolyzer-stacks is merged into a first gas stream, including the following steps: in one step a concentration of impurities, which is originated by a second reaction gas electrochemically produced at a second electrode type of each of the electrolyzer-stacks is determined, within the first gas stream. In another step a trigger signal is generated if the concentration of the impurities of the second reaction gas within the merged first reaction gas exceeds a specific second reaction gas level. In another step at least one electrolyzer-stack out of the plurality of electrolyzer-stacks is identified, which is low performing in respect to excessively feeding second reaction gas impurities into the first gas stream, by measuring a current density of at least one electrolyzer-stack of the plurality of electrolyzer-stacks, if the trigger signal is generated.

### BRIEF DESCRIPTION OF THE SEVERAL VIEWS OF THE DRAWING(S)

FIG. 1 is a calibration curve in accordance with the disclosure.

FIG. 2 is a diagram of a system for electrochemical production of a first reaction gas in accordance with the disclosure.

### DETAILED DESCRIPTION OF THE INVENTION

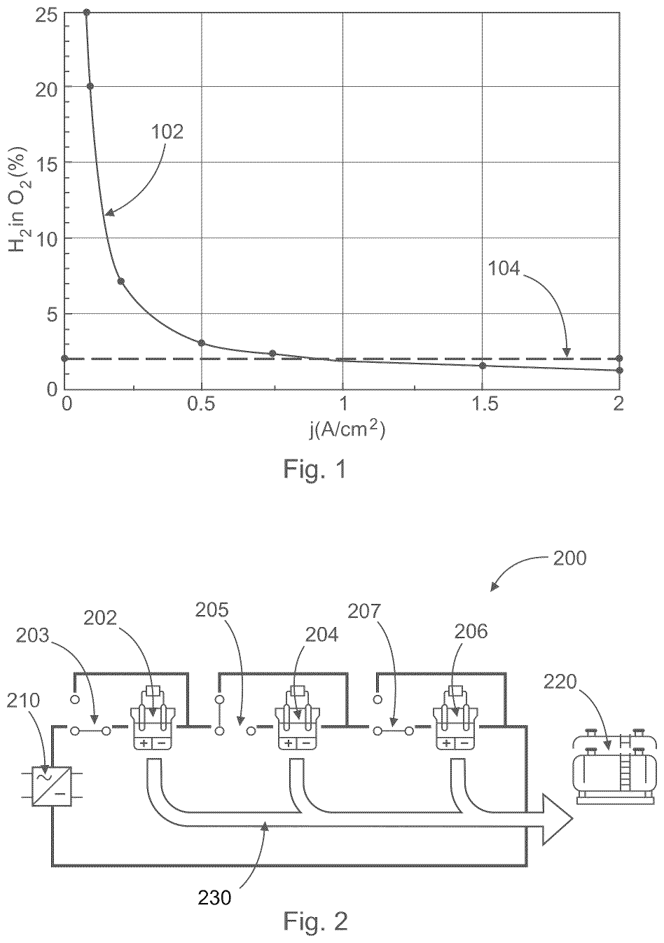

FIG. 1 schematically sketches a calibration curve as a diagram showing the relationship 102 between the current density j measured in A/cm² and a concentration of hydrogen gas in oxygen gas measured in percent. Within the diagram a limit curve 104 is drawn for identification of the critical current density j, wherein the critical current density can be determined where the curve 102 and the limit curve 104 cross each other.

FIG. 2 schematically sketches a system for electrochemical production of a first reaction gas, wherein the first reaction gas can be oxygen gas or hydrogen gas. The system includes an electrical power supply 210 a plurality of electrolyzer-stacks 202, 204, 206 which are configured to be electrically coupled to the power supply 210 in series. Each of the plurality of electrolyzer-stacks 202, 204 and 206 can be disconnected from the electrical power supply 210 by a switch 203, 205, 207 for bypassing the electrical current to the next electrolyzer-stack 202, 204, 206. A first reaction gas which can be electrochemically produced by each of the electrolyzer-stacks 202, 204, 206 are merged into a first gas stream 230, e.g. by a system of pipes, which are fluidly coupled to each of the electrolyzer-stacks 202, 204, 206, and where the first gas stream 230 can be fed into a tank 220, e.g. by means of the system of pipes.

A gas sensitive sensor can be located within the first gas stream 230 and/or the tank 220. The physical gas sensitive sensor can be configured to monitor an amount of oxygen gas in the hydrogen stream if the first gas stream 230 mainly includes hydrogen gas. Alternatively or additionally, if the first gas stream 230 mainly includes oxygen gas the gas sensitive sensor can be configured to monitor an amount of hydrogen gas in the oxygen gas stream. For the case that as well hydrogen gas and oxygen gas is produced by the electrolyzer-stacks, a second pipe system including a second tank, which are not shown here, is connected to the electrolyzer-stacks, which are configured to produce oxygen gas as well as hydrogen gas.

If such the gas sensitive sensor determines an impurity concentration exceeding a threshold value, of e.g. 2% oxygen in hydrogen, the system is configured, e.g. by an electrolyzer-stack operation device, not shown here, to generate a trigger signal or an alarm signal. If the trigger signal is generated the electrolyzer-stack operation device can identify an optimal way to reduce the amount of oxygen impurity within the produced hydrogen gas, while minimizing the impact on a hydrogen production rate, e.g. by bypassing the electrolyzer stack 204, while maintaining the electrolyzer-stacks 202 and 206 in operation as closer described by an example below.

The three electrolyzer-stacks 202, 204, 206 can be configured to produce hydrogen at different production rates and different oxygen impurity levels. The hydrogen gas thus produced by all three stacks 202, 204, 206 can be directed to be collected in one common storage tank 220. As an example, stack 202 has a hydrogen gas production rate of 300 m³/h with 3% of oxygen impurity; stack 204 produces hydrogen gas at a rate of 200 m³/h with 5% of oxygen impurity; and stack 206 produces 800 m³/h of hydrogen containing 1% of oxygen impurity. These hydrogen gas flow, when collected in the storage tank, which is equipped with a physical oxygen sensor, has an overall oxygen gas impurity content of 2.07%, which is above a 2% threshold value 104.

As the trigger signal is generated, the electrolyzer-stack operation device is set up to determine and/or to analyse the current densities of each of the individual stacks 202, 204, 206, comparing the real-time values of the current density with the limit value of the calibration curve 102. The electrolyzer-stack operation device identifies that stacks 202 and 204 are producing hydrogen with an oxygen content above the allowed value of 2%, but the electrolyzer-stack operation device also determines, e.g. by disconnecting the electrolyzer-stack 204, with the lowest current density, that by shutting down the electrolyzer-stack 204, the overall oxygen level in the storage tank is brought down from 2.07% to 1.54%, avoiding the need to shut down the electrolyzer-stack 202.

Therefore, the electrolyzer-stack operation device can operate the switches 203, 205, 207 as shown in FIG. 2, by bypassing electrolyzer-stack 204, while maintaining electrolyzer stacks 202 and 206 in operation, minimizing production rate reduction, while guaranteeing the quality and safety standards.

Similarly, the hydrogen content in the oxygen streams can also be monitored in this way, so that when the threshold value is reached, corresponding corrective actions can be taken.

An electrolyzer-stack can be a stack of electrolyzer-cells, which are stacked and are electrically coupled to each other in respect to electrical power provided to the stack, such that each electrolyzer-cell, which can include an anode-electrode and a cathode-electrode, wherein the anode-electrode and the cathode-electrode being different electrode types are arranged at opposite sides of a membrane, which can act as an electrolyte. An amount of electrolytic generated gas at the anode-electrode and/or the cathode-electrode can be collected from each electrolyzer-cell to build an anode-flow of gas for the anode-electrodes as well as a cathode-flow of gas at the cathode-electrodes of each electrolyzer-stack.

An electrolyzer-stack can produce a first reaction gas electrochemically at the anode-electrodes and different first reaction gas electrochemically at the cathode-electrodes of the electrolyzer-stack at the same time. The first reaction gas can be a reaction product formed by an electrolyze reaction at a respective type of electrode.

The trigger signal can be an alarm or an alarm signal, which is generated if a concentration, which is e.g. determined by a gas sensitive sensor, of the second reaction gas exceeds a specific limit.

A flow of first reaction gas of a plurality of individual electrolyzer-stack can be merged by a system of pipes fluidly connected to each electrolyzer-stack, wherein the pipes are connected accordingly. The merged first gas stream can be collected using a tank, which is connected to the system of pipes accordingly.

According to an aspect, the identification of a low performing electrolyzer-stack by measuring a current density of an electrolyzer-stack can alternatively or additionally be done by measuring a current density of a subsystem of the electrolyzer-stack, as for instance a part of electrolyzer-stack-cells and/or individual electrolyzer-stack-cell.

According to an aspect, the measurement of the current density of the at least one electrolyzer-stack can be triggered by the trigger signal and/or the measurement of the current density can be continuously monitored and values of the measurement can be analyzed, if triggered by the trigger signal.

Using other words, the method provides a solution that instead of monitoring the situation of every electrolysis-stack of the plurality of electrolyzers-stack with a plurality of gas-sensitive sensors, at least a sensor can be located within the first gas stream and/or in a storage tank for the merged and/or collected reaction gas, as for instance hydrogen gas and oxygen gas, produced by several electrolyzer-stacks. If an impurity level, respectively a concentration of the second reaction gas, in the first gas stream and/or the storage tank is within the required safety and quality levels, e.g. less than 2% hydrogen gas in oxygen gas, it is considered that all electrolyzer-stacks feeding the storage tank are operating correctly. If a threshold value of impurities of the second reaction gas within the first reaction case is exceeded, an alarm signal can be triggered, and an algorithm can be used to identify the faulty electrolysis-stack(s).

Once the alarm signal is triggered, a control system, as an electrolyzer-stack operation device, can determine a current density generated by individual electrolysis-stacks, which feeds the storage tank. The determined values of the current density can be used as soft sensors or virtual sensors to determine an impurity concentration, since for each of these electrolyzer-stacks, a calibration curve can be set up upfront, which relates the determined current density of the individual electrolyzer-stack to a concentration of hydrogen gas within the produced oxygen gas.

The hydrogen gas can be originated by a diffusion process from a cathode electrode of a cell of the electrolyzer-stacks to the respective anode electrode of an oxygen side of the cell of the electrolyzer-stack. That means an anodic hydrogen content can be determined by using the calibration curve.

Using an algorithm for the determination of the current density of individual electrolyzers-stacks, by comparing an actual current density with the calibration curve, can be used to identify a faulty or low performing electrolyzer-stack and electrical power provided to that low performing electrolyzer-stack can be bypassed or disconnected.

As a result, the low performing electrolyzer-stack can be serviced, such that the hydrogen gas concentration within a produced oxygen gas can be kept below the safety limit. In addition, for the production of hydrogen gas a concentration of oxygen gas within a produced and collected hydrogen gas can be kept below a required quality level.

Advantageously, based on the pre-existing calibration curves of the electrolyzer-stacks, and amount of anodic hydrogen and cathodic oxygen can be determined for each electrolyzer-stack in a real-time manner, without disconnecting individual electrolyzer-stacks. By measuring an impurity concentration of anodic hydrogen and/or cathodic oxygen within the merged first gas stream within a pipe and/or a tank an early detection system can be set up. As a result, there can be provided a redundancy to physical sensors located in the collecting tanks, were hydrogen gas and oxygen gas is stored, thereby increasing a reliability of the system.

Instead of monitoring the impurity concentration of each electrolysis stack using individual sensors, it is possible to locate at least one sensor within the merged first gas stream and/or a storage tank, where the first gas stream, as the hydrogen gas stream and/or oxygen gas stream produced by several electrolyzer-stacks is collected. If the impurity levels determined by the sensor exceeds a threshold value, an alarm signal or a trigger signal can be generated and an algorithm can be used to identify the faulty electrolysis stacks by measuring the current density of each electrolysis stack, using the determined current density values as a soft sensor.

According to an aspect, the first reaction gas electrochemically produced at the first electrode type is oxygen and the second reaction gas electrochemically produced at the second electrode type is hydrogen. Alternatively or additionally the first reaction gas electrochemically produced at the first electrode type is hydrogen and the second reaction gas electrochemically produced at the second electrode type is oxygen. That means, that oxygen gas as a first reaction gas can be produced at an anode-electrode and/or hydrogen as a first reaction gas can be produced at a cathode electrode.

That means, that with the method described above a hydrogen content in an oxygen stream can be monitored in this way, as well as an oxygen content in a hydrogen stream, such that, if a corresponding impurity level threshold value is reached, corrective actions can be taken. By this, a purity level of oxygen gas within hydrogen gas can be enhanced and by determining a hydrogen level within oxygen gas safety risks can be reduced by this soft sensing.

According to an aspect, the identification of the at least one low performing electrolyzer-stack out of the plurality of electrolyzer-stacks, is done by comparing the measured current density of the at least one electrolyzer-stack with a critical current density. Particularly the at least one low performing electrolyzer-stack can be identified if the measured current density of that electrolyzer-stack is below the critical current density.

That is because a typical relationship between the current density of the electrolyzers-stacks and the impurity level is reverse proportional, which can be shown in particular by means of a polarization curve, at least as an approximation.

According to an aspect, specific second reaction gas level is a hydrogen reaction gas level if the first reaction gas is oxygen. If the specific second reaction gas level is an oxygen reaction gas level the first reaction gas is hydrogen. Particularly the hydrogen reaction gas level and the oxygen reaction gas level can be different.

According to an aspect, a current density of each of several of the plurality of electrolyzer-stacks are measured and each of the measured current densities are compared with an individual critical current density, which is assigned to each of the several electrolyzer-stacks, to identify a single low performing electrolyzer-stack with a measured current density, which is lowest below its individual critical current density, to identify the low performing electrolyzer-stack.

In this respect, the identified low performing electrolyzer-stack can be seen as the electrolyzer-stack producing a highest percentage of impurity, because its current density is most below its individual critical current density.

Using other words, that electrolyzer-stack should be identified which is worst operating in respect of impurities production and particularly this identified electrolyzer-stack can be disabled or shutdown.

According to an aspect, the critical current density is updated during the operation of the plurality of electrolyzer-stacks to account for degradation of the electrolyzer-stacks.

During operation, the membrane can age or degrade, eventually leading to a higher diffusivity of the gases from one electrolytic half-cell of the electrolyzer-stacks to the other. As a result of this, a calibration curve of the electrolyzer-stack can be shifted upwards and/or a critical current density of the electrolyzer-stack can be changed. That means, that with time, e.g. a 2% threshold value of impurities can result from higher current densities, e.g. changing from 1.0 A/cm\hat{ }2 to 1.2 A/cm\hat{ }2.

If a current density is determined at each electrolyzer-stack it enables to individual update the calibration curve, respectively the critical current density, as the electrolytic cells age.

If for instance, a plurality of electrolyzer-stack is in production for two years the calibration curve and/or the critical current density of all or individual electrolyzer-stacks can be updated. For this update, at least time by time or on a regular base, current density and voltage of the individual electrolyzer-stacks are measured, particularly to update the calibration curve and/or the critical current density. Because of operating the electrolyzer-stacks at different operation points, caused by an amount of electrical energy which is provided to the plurality of electrolyzer-stacks over time, a calibration curve can be updated by storing this values. Alternatively or additionally, the production load can be distributed between some of the electrolyzer-stacks of the plurality of electrolyzer-stacks to operate the electrolyzer-stack at different set points and by this the calibration curve and/or the critical current density can be determined. Using other words, a set point for operation of an electrolyzer-stack can be swept over time.

According to an aspect, the individual critical current density for each of the several electrolyzer-stacks is updated during the operation of these electrolyzer-stacks to account for degradation of these electrolyzer-stacks.

According to an aspect, the updated critical current density and/or the updated individual critical current density of the electrolyzer-stacks is determined by a linear relationship between operating hours of at least one electrolyzer-stack and the critical current density of that electrolyzer-stack.

Using other words, on a base of an aging curve for the electrolyzer-stack respectively of the calibration curve, a shift of the calibration curve over time can be used to determine the critical current density, particularly of an individual electrolyzer-stack.

According to an aspect, the updated critical current density and/or the updated individual critical current density of the electrolyzer-stack is determined during operation of the plurality of electrolyzer-stacks by operating at least some electrolyzer-stacks of the plurality of electrolyzer-stacks a different set points.

The current density determines how much Oxygen and Hydrogen is produced at that an individual operating point. That means with other words, by comparing that operating point with a given calibration curve, which can be updated during operation, a crossover, respectively a diffusion of one gas to the other side of a membrane of the electrolyzer-stack can be determined.

By shifting or modifying the operational point of the electrolyzer-stack, the production load of individual electrolyzer-stacks of the plurality of electrolyzer stacks can be shifted. A set point, respectively an operation point, of an electrolyzer-stack can be defined by the operating conditions, mainly determined by a voltage and current provided to the electrolyzer-stack, at which the electrolyzer-stack is operating for producing a specified first reaction gas.

Each electrolyzer-stack can be configured for determining DC and/or AC current and DC and/or AC voltage measurements. Particularly these measurements can be used for Electrochemical Impedance Spectroscopy analysis, which finally allows to determine a degradation of the analysed electrolyzer-stack, to be used as an input and/or to update a calibration curve, based on the result of the Electrochemical Impedance Spectroscopy analysis, after a period of hours of operation.

According to an aspect, the updated critical current density and/or the updated individual critical current density of the electrolyzer-stacks is determined by storing values of voltage and current density of the electrolyzer-stacks during operation of the plurality of electrolyzer-stacks at different set points within a period of operation time.

According to an aspect, the updated critical current density and/or the updated individual critical current density of the electrolyzer-stacks is determined by impedance spectroscopy measurement of at least several electrolyzer-stacks of the plurality of electrolyzer stacks.

Advantageously, adapting the set point for operating the electrolyzer-stacks can be done by the result of the impedance spectroscopy measurements, particularly for determining a Polarization Curve at different Frequencies (Impedance Spectroscopy) or at least at frequencies that are excited by the electric power supply of the electrolyzer-stacks for operation. Particularly low frequency variations can even be triggered by the overall control system of a plant with the plurality of electrolyzer-stacks via the power supply.

According to an aspect, the impedance spectroscopy measurement is performed by using harmonics injected by the electrical power provided to the plurality of electrolyzer-stacks.

According to an aspect, the identified low performing electrolyzer-stack is disconnected from its provided electrical energy.

For this the electrical energy can be bypassed if the plurality of electrolyzer-stacks are electrically connected in series or disconnected, e.g. by opening a switch.

According to an aspect, the above described steps of identifying a single electrolyzer-stack with the lowest current density and disconnecting it from its provided electrical energy can be repeated until the trigger signal is no longer generated.

Advantageously, using this algorithm to identify electrolyzer-stacks to be disconnected can keep a production rate of the first reaction gas with a multitude of electrolyzer-stacks at a high level, by still providing high quality and safety standards.

According to an aspect, the waiting time after an identified electrolyzer-stack is disconnected can be introduced, which is long enough that the system of electrolyzer-stacks can approach a new equilibrium or steady state, before the new measurement of the impurity concentration is performed.

According to an aspect, the concentration of impurities of the second reaction gas is determined by a gas sensitive sensor for generating the trigger signal.

Such a gas sensitive sensor can be located within a tank collecting the merged first gas stream and/or the gas sensitive sensor can be located within a pipe carrying the merged first gas stream before entering the tank.

According to an aspect, the gas sensitive sensor is located for sensing within the first gas stream. Alternatively or additionally the gas sensitive sensor is located for sensing within a tank, wherein the tank is configured in respect to the plurality of electrolyzer-stacks for collecting the first reaction gas of the first gas stream.

An electrolyzer-stack operation device is proposed, including: a current density measurement input, which is configured to receive current density measurement values of each of a plurality of electrolyzer-stacks; and a switch control device, which is configured to be signally coupled to switches of each of the plurality of electrolyzer-stacks, for disconnecting any electrolyzer-stack of the plurality of electrolyzer-stacks from the provided electrical energy; and a trigger signal interface and/or an interface for a gas sensitive sensor; and a control device, particularly including a computer, which is signally coupled with the current density measurement input and to the switch control device and to the first signal interface and/or the interface for the gas sensitive sensor. The control device can be configured to perform a method for operating a plurality of electrolyzer-stacks as described above.

A use of an electrolyzer-stack operation device as described above is proposed, for controlling a plurality of electrolyzer stacks.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for operating a plurality of electrolyzer-stacks, wherein each of the plurality of electrolyzer-stacks are configured to be provided with water and electrical energy to produce at least a first reaction gas electrochemically at a first electrode type of each of the electrolyzer-stacks, and wherein the first reaction gas produced by each of the plurality of electrolyzer-stacks are merged into a first gas stream, the method comprising:
determining a concentration of impurities, which is originated by a second reaction gas electrochemically produced at a second electrode type of each of the electrolyzer-stacks, within the first gas stream;
generating a trigger signal if the concentration of the impurities of the second reaction gas within the merged first reaction gas exceeds a specific second reaction gas level;
identifying at least one electrolyzer-stack out of the plurality of electrolyzer-stacks, which is low performing in respect to excessively feeding second reaction gas impurities into the first gas stream, by measuring a current density of at least one electrolyzer-stack of the plurality of electrolyzer-stacks, when the trigger signal is generated.

2. The method according to claim 1, wherein the first reaction gas electrochemically produced at the first electrode type is oxygen and the second reaction gas electrochemically produced at the second electrode type is hydrogen; or wherein the first reaction gas electrochemically produced at the first electrode type is hydrogen and the second reaction gas electrochemically produced at the second electrode type is oxygen.

3. The method according to claim 1, wherein the identification of the at least one low performing electrolyzer-stack out of the plurality of electrolyzer-stacks is done by comparing the measured current density of the at least one electrolyzer-stack with a critical current density.

4. The method according to claim 3, wherein the method includes identifying the at least one low performing electrolyzer-stack when the measured current density is below the critical current density.

5. The method according to claim 2, wherein the specific second reaction gas level is a hydrogen reaction gas level when the first reaction gas is oxygen; and wherein the specific second reaction gas level is an oxygen reaction gas level when the first reaction gas is hydrogen; and wherein particularly the hydrogen reaction gas level and the oxygen reaction gas level are different.

6. The method according to claim 3, wherein a current density of each of several of the plurality of electrolyzer-stacks is measured and each of the measured current densities are compared with an individual critical current density, which is assigned to each of the several electrolyzer-stacks, to identify a single low performing electrolyzer-stack with a measured current density, which is lowest below its individual critical current density, to identify the low performing electrolyzer-stack.

7. The method according to claim 1, wherein the critical current density is updated during operation of the plurality of electrolyzer-stacks to account for degradation of the electrolyzer-stacks.

8. The method according to claim 7, wherein the individual critical current density for each of the several electrolyzer-stacks is updated during the operation of these electrolyzer-stacks to account for degradation of these electrolyzer-stacks.

9. The method according to claim 7, wherein the updated critical current density and/or the updated individual critical current density of the electrolyzer-stacks is determined during operation of the plurality of electrolyzer-stacks by operating at least some electrolyzer-stacks of the plurality of electrolyzer-stacks a different set points.

10. The method according to claim 9, wherein the updated critical current density and/or the updated individual critical current density of the electrolyzer-stacks is determined by impedance spectroscopy measurement of at least several electrolyzer-stacks of the plurality of electrolyzer stacks.

11. The method according to claim 10, wherein the impedance spectroscopy measurement is performed by using harmonics injected by the electrical power provided to the plurality of electrolyzer-stacks.

12. The method according to claim 1, wherein the identified low performing electrolyzer-stack is disconnected from its provided electrical energy.

13. The method according to claim 12, which is repeated until the trigger signal is no longer generated.

14. The method according to claim 1, wherein the concentration of impurities of the second reaction gas is determined by a gas sensitive sensor for generating the trigger signal.

15. The method according to claim 10, wherein the gas sensitive sensor is located for sensing within the first gas stream.

16. The method according to claim 10, wherein the gas sensitive sensor is located for sensing within a tank, and wherein the tank is configured in respect to the plurality of electrolyzer-stacks for collecting the first reaction gas of the first gas stream.

17. An electrolyzer-stack operation device, comprising:
a current density measurement input, which is configured to receive current density measurement values of each of a plurality of electrolyzer-stacks;
a switch control device, which is configured to be signally coupled to switches of each of the plurality of electrolyzer-stacks, for disconnecting any electrolyzer-stack of the plurality of electrolyzer-stacks from provided electrical energy;
a trigger signal interface and/or an interface for a gas sensitive sensor; and
a control device that is signally coupled with the current density measurement input; and signally coupled to the switch control device; and signally coupled to the first signal interface and/or the interface for the gas sensitive sensor;
wherein the control device is configured to perform a method, the method comprising:
determining a concentration of impurities, which is originated by a second reaction gas electrochemically produced at a second electrode type of each of the electrolyzer-stacks, within the first gas stream;
generating a trigger signal if the concentration of the impurities of the second reaction gas within the merged first reaction gas exceeds a specific second reaction gas level;
identifying at least one electrolyzer-stack out of the plurality of electrolyzer-stacks, which is low performing in respect to excessively feeding second reaction gas impurities into the first gas stream, by measuring a current density of at least one electrolyzer-stack of the plurality of electrolyzer-stacks, when the trigger signal is generated.
